(19) United States
(12) Patent Application Publication
(10) Pub. No.: US 2025/0186835 A1
(43) Pub. Date: Jun. 12, 2025

(54) METHOD FOR ESTIMATING FITNESS SCORES FROM WEARABLE DEVICE DATA

(71) Applicant: SAMSUNG ELETRÔNICA DA AMAZÔNIA LTDA., CAMPINAS (BR)

(72) Inventors: Thomas Beltrame, CAMPINAS (BR); Maiko Min Ian Lie, CAMPINAS (BR); Eduardo Kazuharu Chikui, CAMPINAS (BR); Vinícius Torres Dutra Maia da Costa, CAMPINAS (BR); Renato Stoffalette João, CAMPINAS (BR); Marcus de Assis Angeloni, CAMPINAS (BR); Paula Ramos Pinto, CAMPINAS (BR); Juscelino Lopes de Oliveira Junior, CAMPINAS (BR); Hyun Gi Ahn, Suwon-si (KR); Kyungsub Min, Suwon-si (KR); DongHyun Roh, Suwon-si (KR); Jinmook Lim, Suwon-si (KR)

(73) Assignee: SAMSUNG ELETRÔNICA DA AMAZÔNIA LTDA., CAMPINAS (BR)

(21) Appl. No.: 18/532,628

(22) Filed: Dec. 7, 2023

(51) Int. Cl. A63B 24/00; G06N 5/022; G16H 20/30; G16H 40/63

(52) U.S. Cl. CPC A63B 24/00; G06N 5/022; G16H 20/30; G16H 40/63; A63B 2024/0065; A63B 2024/0068

(57) ABSTRACT

An automatic method of prediction for health-related physical fitness status in a non-invasive manner by only gathering data from sensors embedded in wearable devices. In contrast to previous approaches, the present invention solution provides a complete characterization of fitness status by estimating all five health-related fitness domains defined by the ACSM.

### TECHNICAL FIELD

The present invention is related to the characterization of health-related physical fitness (HRPF) based on estimates of all HRPF components defined by the American College of Sports Medicine (ACSM). These estimates can be combined to create an overall fitness status (OFS) indicator with many applications, including a better characterization of user health status, personalized training prescriptions, and early identification of chronic diseases. The main goal of the present invention is to estimate a holistic fitness measure from different physiological domains.

### BACKGROUND OF THE INVENTION

Currently, low physical activity is the fourth leading risk factor for mortality worldwide. On the other hand, medical equipments are very expensive, large and cumbersome, which makes them impossible to be acquired by the general population for constant health monitoring. Moreover, they require specialized knowledge and are impractical to be used by ordinary users. Even though there is limited evidence suggesting that is possible to improve health by using wearable fitness trackers and wearable devices, these devices are very popular and can indeed help users to monitor daily activities and produce affordable physical fitness information.

Continuous fitness status monitoring is beneficial for tracking physical training/treatment progression as well as health condition. Since the onset of chronic diseases are most of the time related to a decrease in fitness status, it is essential for early identification of sub-clinical trace of chronic illnesses including, but not limited to, cardiovascular diseases and diabetes, which account for three quarters of all deaths around the world and impose high economic burdens. Continuous biological data from wearable devices can be modeled to estimate the fitness status, thus enabling it to be tracked in a cost-effective way that is more accessible than using specialized clinical equipment, which is costly and requires the support of health professionals. Current commercial solutions can estimate fitness levels of some health-related domains such as cardiovascular and muscular strength.

Cardiorespiratory health indicators have been used to predict mortality as well as for fitness recommendations. However, none of the current solutions have been capable of providing multi-domain fitness levels prediction across all the five health-related domains defined by the ACSM, or normalization based on group-specific distributions. According to the American College of Sports Medicine (ACSM), health related physical fitness (HRPF) is not a single entity but rather a physiological measure composed of five measurable components, i.e., muscular strength, muscular endurance, flexibility, cardiorespiratory endurance, and body composition.

The protocols to evaluate an individual fitness level are usually performed under the supervision of health experts and by using specific medical equipment. One way to perform the fitness tests is to use direct measurement of cardiorespiratory responses during maximal exercise tests, collect the body composition information with a bio-impedance scale, etc. There are other methods to assess the fitness levels indirectly under submaximal exercises, but both the former and the latter require specialized professionals and equipment to monitor and conduct the supervised experiments.

With the current advances in the field of artificial intelligence as well as cutting-edge technologies of embedded sensors at hand, biological signals can be monitored and used to predict important health indicators in a non-invasive, unobtrusive, and constant manner.

For example, in the paper entitled “Ensembled artificial neural networks to predict the fitness score for body composition analysis” published in 2011 by Cui et al., the authors used the Inbody 720 to measure 139 body composition variables from 1227 volunteers and trained an ensemble of artificial neural networks to predict the nutrition and health status of staff and students in Yuan Ze University and to select influential variables from the total body composition variables. In that work, they claimed to have found a subset of most important variables to train ensembled artificial neural networks and predict the body composition score with the objective of helping nutritionists to improve the health condition of staff and students in the university. They proposed a solution to simply predict the body composition score after training EANN with data collected from the Inbody 720. However, the present invention does not require any external equipment as the data is collected constantly in the wearable device and produces the fitness levels on the fly.

The paper entitled “Estimation of Health-Related Physical Fitness (HRPF) Levels of the General Public Using Artificial Neural Network with the National Fitness Award (NFA) Datasets” published in 2021 by Lee et al., describes an artificial neural network model to predict health-related physical fitness of the general population. However, they used publicly available data from the National Fitness Award (NFA) of the Korean Sports Promotion Foundation (KSPO) and limited their prediction to the HRPF characteristics of the general public in South Korea. Moreover, they predicted the direct metrics, not the scores, for each of the domains, namely flexibility, muscular strength, cardiorespiratory endurance and muscular endurance. Their approach differs from the present invention's as they proposed the prediction of the values in absolute units for each of the fitness domains using data not necessarily collected from wearable devices but also from laboratory tests, wherein the present invention uses the prediction of one more domain, i.e., body composition as well as a holistic view of the five domains that represent an overall fitness status of an individual using non-supervised data from continuous reads on wearable devices.

The patent document JP 7051704 B2, entitled “Information processing method and information processing equipment”, published on Nov. 4, 2022, by Sony Communications, describes a technology that performs machine learning using data acquired from wearable devices, such as user profile information (e.g., height, weight, sex) and vital data (e.g., body temperature, blood pressure, pulse, SpO2) to predict fitness values (muscle strength, agility, cooperation, reaction speed and endurance). These values are used to suggest meal content, sleeping time, and amount of exercise. In contrast to the present invention, which predicts all five health-related fitness domains, the only health-related fitness domains predicted by this technology are muscular strength and endurance, while the other values that it predicts are related to skill-related domains (which do not reduce risk of mortality, and thus are not health-related).

The patent document KR 10-2022-0158898 A, entitled “System and Method for Analysing User's Mood by Collecting User's Bio-Activity Data”, published on Dec. 2, 2022, by Disc Cry, describes a system that analyzes user input information (e.g., sex, age, weight, height, current diseases) and bioactivity data (e.g., heart rate, number of steps, calories burned, exercise record, blood pressure) to analyze the user's emotional state and provide suitable exercise and diet prescriptions. While this system processes user input and bioactivity data, it does not address health-related fitness domains, which is the core of the present invention. Moreover, since the exercise and diet prescriptions from this system are not based on estimates of health-related fitness, they are of more limited relevance to health improvement.

The patent document JP 2022-076748 A1, entitled “Information processing method and program”, published on May 20, 2022, by SPLINK INC, describes a system that processes a wide range of health-related information, including user attribute information (e.g., sex, age), lifestyle information (e.g., exercise, drinking, smoking, sleeping), information about living environment (e.g., information about working hours, satisfaction, working style, family structure), test results performed at medical institutions, treatment details, rehabilitation details, medical examination data regarding medications being taken, information on the user's own or family history (genetic information), among others. This data is processed using a dimensionality reduction technique, such as Principal Component Analysis and multiple regression analysis, to reduce the data to a two-dimensional representation which is then used to cluster the data and depict it as a “health map”. The present invention differs from this system in several aspects. Firstly, it provides predictions from data acquired from wearable devices in a non-intrusive manner. Moreover, this data is used to predict all health-related fitness domains, which are well-established and grounded on the sports medicine literature, as opposed to latent variables from dimensionality-reduction approaches. Lastly, as a consequence of being better grounded on sports science and having concrete physiological meaning, the present invention's multidimensional representation is more relevant to health professionals and interpretable to end-users than the aforementioned health map.

As it can be seen from prior art documents, estimates of individual fitness domains, for example the cardiorespiratory domain, provide an oversimplified characterization of the user's health-related fitness status. As such, analysis based on individual fitness domains cannot consider their interaction effects.

Bearing this in mind, the main goal of the present invention is to estimate fitness levels on all the different domains defined by the ACSM to provide a complete and interpretable characterization of health. The proposed invention is based on estimation from biological data and basic user input data collected by smart devices. These estimates provide a complete characterization of the user's health status based on all health-related fitness domains, in contrast to previous approaches that estimate a single or a subset of domains.

In addition to providing a more comprehensive depiction of health status compared to alternative solutions, estimates on these domains are widely interpretable to health professionals since they are well-established in sports science and are relativized by age and sex group into a unit-free standardized score.

### SUMMARY

A method of estimating fitness scores from data associated with a wearable device comprises receiving user profile data comprising: age, gender, weight, height and body mass index; extracting sensor data, which includes bioelectrical impedance analysis (BIA) data, exercise session data and activity level data; obtaining a data set of sensor data features and respective performance on tests for health-related physical fitness (HRPF) domains, including: muscular endurance, muscular strength, flexibility, body composition and cardiorespiratory; normalizing training of the data set by subtracting a mean and dividing by a standard deviation of each feature; obtaining a prediction model for each HRPF domain by training machine learning algorithms, one for each domain, using the normalized data set; and obtaining predictions for a new data instance. The obtaining predictions may be by a) normalizing a new data feature vector by subtracting the mean and dividing by the standard deviation of each variable in a training set; b) applying regression models with source-dependent feature selection and latent variable projection to obtain a prediction for each domain; and c) normalizing each prediction by a distribution corresponding to age and sex of the new data instance.

### BRIEF DESCRIPTION OF THE DRAWINGS

The objectives and advantages of the invention will become clearer through the following detailed description of the example and non-limitative drawings presented at the end of this document.

FIG. 1 depicts the sources of input data and subsequent preprocessing operation(s), including imputation and temporal data aggregation according to an exemplary embodiment of the present invention.

FIG. 2 demonstrates how activity level data is aggregated over time according to an exemplary embodiment of the present invention.

FIG. 3 demonstrates how exercise session data is aggregated over time according to an exemplary embodiment of the present invention.

FIG. 4 illustrates the overall architecture of the domain score predictors according to an exemplary embodiment of the present invention.

FIG. 5 demonstrates the effect of the normalization on scale and unit according to an exemplary embodiment of the present invention.

FIG. 6 demonstrates the effect of the normalization on score comparability according to an exemplary embodiment of the present invention.

FIG. 7 illustrates how each domain score is grouped into a representation of multi-domain fitness status according to an exemplary embodiment of the present invention.

FIG. 8 shows a comparison of single-domain and multi-domain fitness status characterization according to an exemplary embodiment of the present invention.

FIG. 9 demonstrates how the proposed multi-domain fitness status solution can be used to track progress towards a target fitness profile across time according to an exemplary embodiment of the present invention.

### DETAILED DESCRIPTION

Considering the recent interest in health and well-being aligned with cutting-edge artificial intelligence and embedded sensors technologies, the present invention proposes an automatic method of prediction for health-related physical fitness status in a non-invasive manner by only gathering data from sensors embedded in wearable devices. In contrast to previous approaches, the present invention solution provides a complete characterization of fitness status by estimating all five health-related fitness domains defined by the ACSM.

Furthermore, the results were validated in a clinical trial executed under the supervision of health specialists following the ACSM guidelines. The idea described in this document is composed of different regression models trained with demographic and physiological data collected from a wearable device. Some of the features were read directly from the wearable device, while others were carefully engineered (such as body surface and physical-activity status) after extensive experimental analysis. All the features that were used demonstrated importance for the health-related fitness indicator predictions. The engineered features as well as the features that were read directly from the wearable device were compiled into a single dataset, preprocessed and input into a pipeline to predict the HRPF levels for all five health-related fitness domains: muscular strength, muscular endurance, flexibility, cardiorespiratory endurance, and body composition. Each model was trained to predict a specific target value, i.e. the ground truth obtained by the ACSM's recommended standard test for each domain. The predicted data was then normalized by the expected ACSM normal values according to user's age and sex.

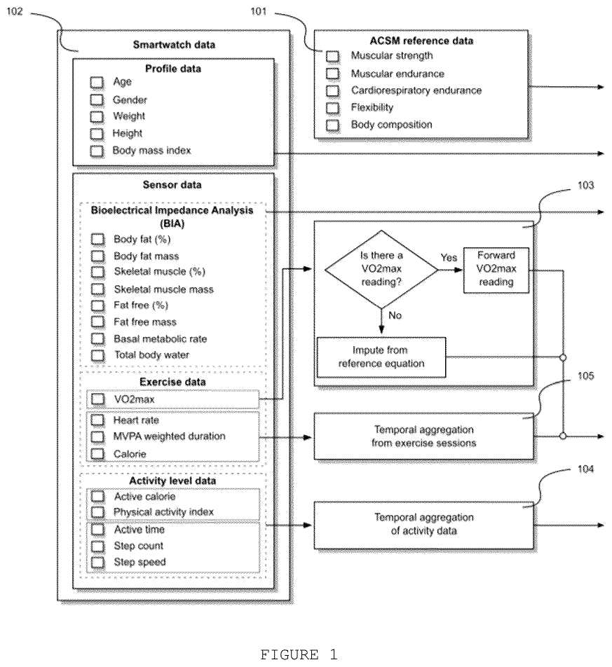

As depicted in FIG. 1, the present invention operates using ACSM reference data (ACSM normative values) (101) and smartwatch data (wearable device data) (102) as input. The former specifies distributions of the output variables (i.e., HRPF test performances) for different age and sex groups, which are used to convert raw test values into relativized scores. The latter is composed of values that can be categorized as user profile, bioelectrical impedance analysis (BIA), exercise, and activity level data. User profile data is specified by the user, BIA data is collected when the user performs the BIA function of the wearable device, while VO2max data (103) may be optionally provided by the wearable device if available, otherwise, it is estimated using an empirical reference equation:

$$\dot{V} {O}_{2 - max} = 79.9 - ( 0.39 \times Age ) - ( 13.7 \times Gender [ 0 = male , 1 = fema l e ] ) - ( 0.127 \times Weight [ lbs ] ) .$$

Since BIA and VO2max data have low daily variability, the last valid values available are used. Temporal aggregation from exercise sessions (Activity level) (104) and Temporal aggregation of activity data (exercise data0 (105), on the other hand, not only have substantial daily variability, but might be noisy, sparse, and high dimensional, so measurements made in the last 30 days are aggregated to obtain informative scalar features. Note that despite its different treatment, VO2max is still categorized as an exercise variable for later operation(s) of the present invention.

Additionally, the feature set was also enriched with exercise session duration weighted by Moderate to Vigorous Physical Activity (MVPA) class, i.e., 4×, 2×, and 1× the session duration if the intensity is very vigorous, vigorous, and light/moderate, respectively, and the Physical Activity Index (ratio of total to resting calories). Combined aggregations on multiple time scales are employed, which differ depending on the source data.

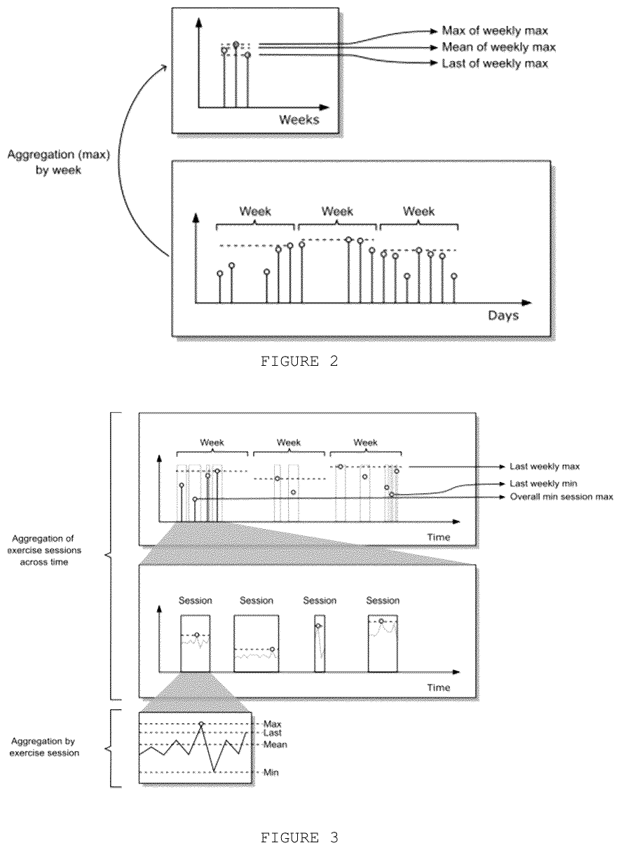

FIG. 2 describes how activity level is aggregated across different time scales within the last 30 days, illustrating how aggregations are composed. The example shows how the mean of weekly maximums of a variable is aggregated, but any aggregation function, such as minimum, maximum, mean, variance, and last, can be used. Aggregation of exercise data is similar. In this case, the data is initially acquired as fragmented in exercise sessions, which might be sparse and non-uniform in size due to the inherent uncertainty of exercise habits during free-living. Each exercise session is aggregated once and then further aggregated as with activity level data, for instance, by week or through the entire 30-day window. FIG. 3 shows an example in which exercise sessions are aggregated by their maximum, and these values can be subsequently subject to several other types of aggregation.

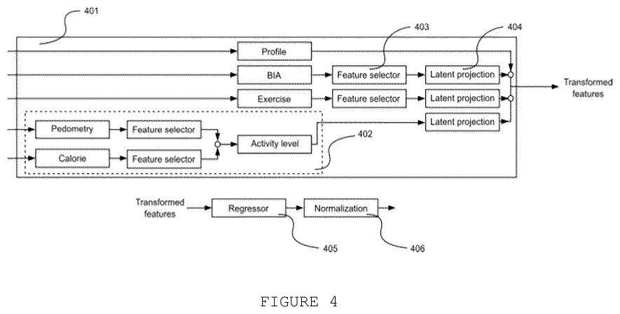

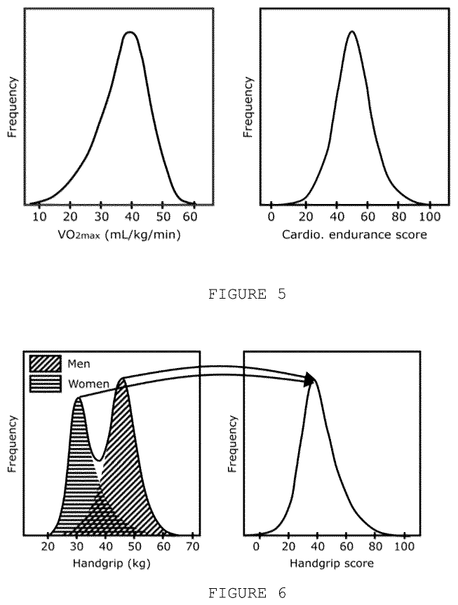

The features extracted by the process described previously are z-score normalized (i.e., each variable is subtracted by the mean and divided by the standard deviation of its values in the training dataset) and forwarded to five estimation models, one for each health-related fitness domain, which follow the same architecture, shown in FIG. 4. As the present invention is trained to predict standardized fitness tests from wearable device data, it requires specialized clinical trials, which are costly. This leads to scarce training data and, consequently, technical limitations, such as the use of lower capacity models (i.e., less parameters) and smaller input dimensionality to avoid overfitting. The architecture of the predictors was designed to address these issues.

More specifically, the predictors follow a data-driven architecture that performs source-dependent feature selection and latent variable projection (401). It has four branches, one for each type of source of input data. User profile features are forwarded directly, while BIA and exercise features are subject to their respective feature selection and latent projection branches. Activity level data also has a dedicated branch (402) with its own latent projection, but its feature selection is performed before since it has subcategories (i.e., pedometry and calorie data) which lead to more consistent feature selection when performed individually.

The feature selector (feature selection mechanism) (403) is based on Pearson correlation with the target variable. It has two main operations: i) dropping features with correlation lower than a threshold (0.2 was effective in the experiments), ii) for each temporal; input variable (e.g., active calorie, step count), forwarding the aggregation with largest correlation. Features that are not temporal (i.e., BIA) and have correlation above the threshold are just forwarded. This process enables leveraging multiple aggregation approaches without increasing dimensionality unnecessarily, which could potentially lead models to overfit.

The latent projection (404) projects features onto a lower dimensional latent subspace using PCA (Principal Component Analysis), which transforms the data by representing each variable as a linear combination of the original input variables that has maximum explained variance. More specifically, its denoising properties are leveraged and the features are projected onto the smallest subset of vectors that is capable of representing a certain fraction of the total variance of the training set, which effectively reduces data dimensionality and discards sources of small variation in the data. However, unlike the naïve approach of performing a single projection for all features, the present invention approach performs source-dependent projections. This is motivated by the fact that data from different sources have different characteristics, such that discarding variables based on correlation and proportion of explained variance from data with heterogeneous characteristics might be too overzealous and discard discriminative information. It is also important to note that the definition of what variables are grouped into specific branches is not arbitrary. They are the result of how the device exposes its data and how historical data is aggregated.

The source-dependent latent projections are concatenated and input to a regressor (linear regression) (405). For the muscular endurance domain, a Poisson regression is employed since the target variable is based on count data (i.e., maximum number of push-ups), which has a skewed distribution that is addressed more adequately by this type of regression. The remaining domains employ Lasso regression, which is a linear regression regularized to perform variable selection by means of incentivizing zero coefficients.

The predicted outputs are estimates of the gold standard for their respective fitness domains. Consequently, they also have domain-specific ranges and units. To provide normalized scores that are more interpretable to end-users and clinically relevant, the output of the predictors is normalized (406) according to group-specific (age and sex) distributions defined by the ACSM (101). More specifically, the normalization f(x) is made according to the following equation:

$$f ( x ) = 100 \frac{x - {P}_{L \%}}{{P}_{H \%} - {P}_{L \%}} ,$$

where x is the value to be normalized, P_{L} % and P_{H} % are the percentiles of the group-specific distribution. L and H are defined by what is provided by the ACSM guidelines, for this specific case, the percentiles are P_{10%} and P_{90%} for flexibility and P_{5%} and P_{95%} for the other domains. One advantage of this normalization is that it presents the fitness levels of each domain as a score (from 0 to 100) that is more understandable for end-users, while remaining clinically relevant since it is based on ACSM criteria and distributions. This is illustrated in FIG. 5, where the distribution of cardiorespiratory fitness levels, which range from approximately 10 to 60 ml/min/Kg, is converted to a score between 0 and 100. A normalized score is easier for an end-user to understand than domain-specific values and units (e.g. ml/min/Kg).

Another advantage of this normalization is the fact that it is made according to age and sex groups. This means that different groups, which have expected different fitness levels in raw values, are mapped to scores that are more meaningful and comparable within their group. For instance, the distribution of handgrip, in Kg, is bimodal, as depicted in FIG. 6. This means that the most common values for men and women are different, thus women would have lower values than men most of the time. By normalizing using group-specific distributions, these raw values are converted into scores that are more meaningful according to their respective groups. For instance, the most common value for men and women are mapped to the most common score in this fitness domain. In summary, normalization into scores makes the values more meaningful and interpretable.

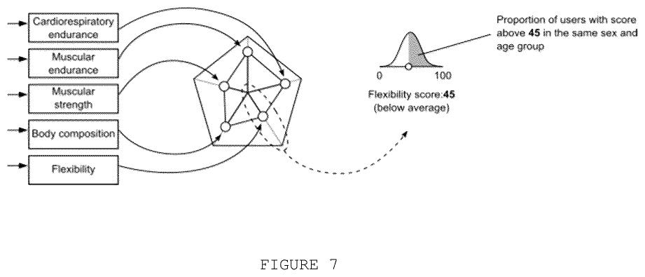

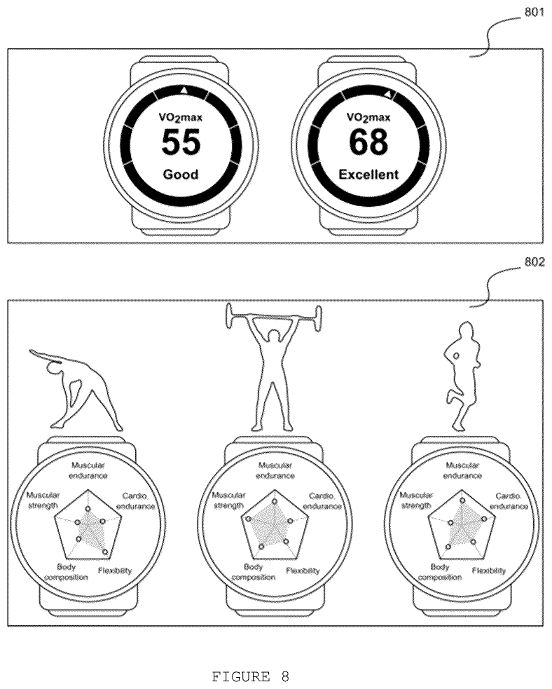

The prediction of scores for all five health-related fitness domains provides a comprehensive description of the user's health, which can be used to present a user-friendly summary adequate for small displays, such as the ones on wearable devices, as shown in FIG. 7.

Therefore, the method proposed by the present invention for estimating fitness scores from wearable device data comprises:
receiving user profile data comprising: age, gender, weight, height and body mass index;
feature extracting sensor data, which includes bioelectrical impedance analysis (BIA) data, exercise session data and activity level data;
obtaining a data set of sensor data features and their performance on tests for health-related physical fitness (HRPF) domains, including: muscular endurance, muscular strength, flexibility, body composition and cardiorespiratory;
normalizing the training data set by subtracting the mean and dividing by the standard deviation of each feature;
obtaining a prediction model for each HRPF domain by training machine learning algorithms, one for each domain, using the normalized data set; and obtaining predictions for a new data instance by:
a) normalizing the new data feature vector by subtracting the mean and dividing by the standard deviation of each variable in the training set;
b) applying regression models with source-dependent feature selection and latent variable projection to obtain a prediction for each domain;
c) normalizing each prediction by the distribution corresponding to the age and sex of the instance.

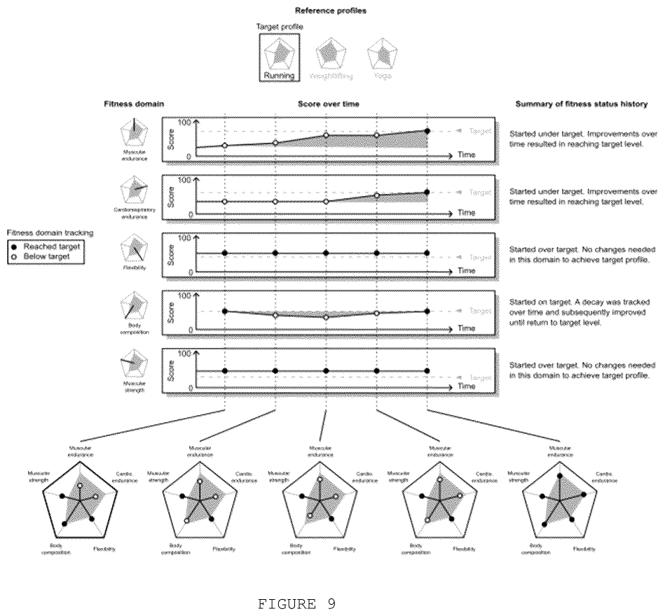

In contrast to commercial alternatives that provide only estimates of a single, or a few, health-related fitness domains (801), the description proposed in the present invention is based on all health-related fitness domains and thus can be used to associate the user to his or her most likely fitness profile across several modalities, enabling straightforward monitoring of specific fitness goals (802) with a specificity that is not possible with less comprehensive alternatives. An example of this multi-dimensional summary and its association to likely fitness profiles is shown in FIG. 8, in which it is contrasted to an alternative approach based on fewer health-related fitness domains.

A group of volunteers was asked to wear a smartwatch for a total period of 30 days in order to collect data for the experimental setup. All the participants were asked to perform at least one running activity every 2 weeks to obtain the maximal oxygen uptake (VO2max in ml/min/kg), and to take the Bioelectrical Impedance Analysis (BIA) measure from the wearable device (to obtain the body fat in %). Following the ACSM standards and guideline, the participants performed the handgrip, push up, sit-and-reach, standard BIA and maximal treadmill exercise tests in the laboratory to obtain the ground truth values that were used as labels to train machine learning models. After data curation, it ended up with data from 104 participants (56 males and 48 females, average age of 36 years old±7 years, weight of 77.6 kg±17.1 kg and height of 168.8 cm±9.2 cm) that was used to train supervised machine learning models to estimate the fitness level for the muscular strength, muscular endurance, flexibility, cardiorespiratory and body composition domains.

In the embodiment of the described invention a smartwatch was used to read biological signals as well as to collect the demographic data input by the user to estimate one's overall fitness status. The method is based on machine learning models and can estimate the user's fitness levels in all different health-related domains, namely muscular strength, muscular endurance, flexibility, cardiorespiratory and body composition. Moreover, the predicted values are normalized by the expected ACSM's normal values according to user's age and sex groups.

The proposed solution can be employed when using other types of wearables equipped with appropriate sensors and microprocessors, such as tablets, smart shirts, and smart rings, as long as they are also constantly reading biological signals from the users and feeding this information to a device with a touchscreen display.

The invention presented in this document proposed a continuous, unobtrusive, and automatic solution for estimating the fitness level of the body as a whole (i.e., at multiple domains of fitness). This solution benefits the user by increasing the specificity of the physical training programs, as well as allowing the users to track how their fitness is progressing through time solely by wearing a wearable device. The algorithms are based on machine learning models that have anthropometric and sensor-based features as inputs. When users track their fitness status using on multidimensional representation, which is based on predictions of all health-related fitness domains, it is straightforward to identify at a glance the domains that require improvement according to what the ACSM considers the normal values for the user's age and sex (e.g., a score below 50 means that the user is below average compared to his/her respective age and sex group). Thus, users can tailor training more accurately to improve fitness according to target goals, such as increasing scores on domains related to specific sports, improving on domains in which the user is more saliently deficient, or to ensure an overall balanced improvement. An example of this use case is shown in FIG. 9, in which a target profile is selected among several associated with specific sports, and training is focused on the development of specific fitness domains to reach a target profile. While approaches based on an individual, or even a few, fitness domains can be useful, only a complete characterization of health-related fitness is enough to provide insights as accurate and comprehensive as the present invention.

Therefore, the present invention provides easy-to-understand information to the final user, simplifying the understanding on how users can improve their health-related fitness level.

Although the present invention has been described in connection with certain preferred embodiments, it should be understood that it is not intended to limit the disclosure to those particular embodiments. Rather, it is intended to cover all alternatives, modifications and equivalents possible within the spirit and scope of the disclosure as defined by the appended claims.

What is claimed is:

1. A method of estimating fitness scores from data associated with a wearable device comprising:
receiving user profile data comprising: age, gender, weight, height and body mass index;
extracting sensor data, which includes bioelectrical impedance analysis (BIA) data, exercise session data and activity level data;
obtaining a data set of sensor data features and respective performance on tests for health-related physical fitness (HRPF) domains, including: muscular endurance, muscular strength, flexibility, body composition and cardiorespiratory;
normalizing training of the data set by subtracting a mean and dividing by a standard deviation of each feature;
obtaining a prediction model for each HRPF domain by training machine learning algorithms, one for each domain, using the normalized data set; and
obtaining predictions for a new data instance by:
a) normalizing a new data feature vector by subtracting the mean and dividing by the standard deviation of each variable in a training set;
b) applying regression models with source-dependent feature selection and latent variable projection to obtain a prediction for each domain; and
c) normalizing each prediction by a distribution corresponding to age and sex of the new data instance.

2. The method as in claim 1, wherein the extracting of sensor data further comprises:
obtaining a most recently valid value within 30 days for VO2max and bioelectrical impedance;
temporally aggregating activity level data by performing multiple aggregations across different time scales; and
temporally aggregating exercise session data features by performing multiple aggregations across different time scales.

3. The method as in claim 2, wherein based on VO2Max data being unavailable, estimating the VO2Max data according to an equation as follows:
$$\dot{V} {O}_{2 - max} = 79.9 - ( 0.39 \times Age ) - ( 13.7 \times Gender [ 0 = male , 1 = fema l e ] ) - ( 0.127 \times Weight [ lbs ] ) .$$

4. The method as in claim 1, wherein the sensor data from the wearable device is an input and user performance on HRPF tests are ground truth for the prediction model.

5. The method as in claim 1, wherein the obtaining of the prediction model for each HRPF domain by training machine learning algorithms further comprises:
source-dependent feature selection, in which bioelectrical impedance analysis (BIA), pedometry, calorie, and exercise session data features are first thresholded according to corresponding Pearson correlation to a target variable, and temporal features are further subject to selection of an aggregation with largest correlation;
source-dependent latent projection, in which profile data, BIA, activity level data and exercise session data forwarded by source-dependent feature selection are subject to their respective Principal Component Analysis (PCA) projection, wherein a smallest subset of vectors representing a given proportion of a variance in the training set is preserved; and
linear regressions on features transformed via source-dependent latent projection, where muscular endurance domain employs Poisson regression, and remaining domains employ Lasso regression.

6. The method as in claim 1, wherein the normalization of a prediction further comprises:
obtaining percentiles of a target variable, according to American College of Sports Medicine (ACSM), for the age and sex of the predicted respective instance; and
for each domain, calculating multiple health-related physical fitness domains' scores of the prediction by subtracting a lowest percentile and dividing by a difference between a highest and lowest percentile,
wherein the normalization of f(x) follows:
$$f ( x ) = 100 \frac{x - {P}_{L \%}}{{P}_{H \%} - {P}_{L \%}} ,$$
where x is a value to be normalized, PL % and PH % are the percentiles of a group-specific distribution, L and H are defined by ACSM guidelines for each domain.

7. The method as in claim 1, further comprising:
displaying, on a display of the wearable device, a simultaneous depiction of multiple health-related physical fitness domains' scores.
